(19) United States
(12) Patent Application Publication
(10) Pub. No.: US 2020/0038616 A1
(43) Pub. Date: Feb. 6, 2020

(54) MULTI-ADJUSTABLE MULTI-STRAP OXYGEN MASK

(71) Applicant: Leanne M. Neace, Lumber Bridge, NC (US)

(72) Inventor: Leanne M. Neace, Lumber Bridge, NC (US)

(21) Appl. No.: 16/527,797

(22) Filed: Jul. 31, 2019

Related U.S. Application Data
(60) Provisional application No. 62/712,252, filed on Jul. 31, 2018.

(51) Int. Cl. A61M 16/06; A61M 16/20; A61M 16/10; A61M 16/00

(52) U.S. Cl. CPC A61M 16/0683; A61M 16/208; A61M 2205/0216; A61M 16/105; A61M 16/0078; A61M 16/1005

(57) ABSTRACT

The subject matter described herein includes a mask for use in providing oxygen or nebulized medicaments. The mask includes at least two straps and a plurality of apertures defined along two sides of the mask. The apertures are configured for receiving the at least two straps at more than two positions along a length of the mask. The mask includes at least one exhalation port and an inhalation port for receiving the oxygen or nebulized medicaments. In operation an oxygen delivery tube and reservoir bag are connected to the inhalation port.

### CROSS REFERENCE TO RELATED APPLICATIONS

This application claims the benefit of U.S. Provisional Application No. 62/712,252, titled MULTI-ADJUSTABLE MULTI-STRAP OXYGEN MASK, filed Jul. 31, 2018, which is herein incorporated by reference in its entirety.

### TECHNICAL FIELD

The presently disclosed subject matter is directed towards an adjustable oxygen mask. Specifically, the presently disclosed subject matter is directed towards an adjustable oxygen mask with multiple points of adjustment having at least two straps.

### BACKGROUND

The use of oxygen masks is ubiquitous in hospital and home care settings. Often patients suffer when oxygen masks slide down a patient's face, putting pressure and causing shearing wounds on the bridge of the nose. Single strap oxygen masks cause irritation when the strap is stretched tightly behind the patient's ears. Elderly patients often experience the breakdown of skin when subjected to poorly fitting and sliding masks for prolonged periods of time.

What is needed is a mask for proving a stable, more comfortable means of supplying oxygen and nebulized medication to patients. The presently disclosed subject matter provides improved systems and methods of manufacturing and providing a mask that overcomes the disadvantages of the prior art.

### SUMMARY

This summary is provided to introduce, in a simplified form, concepts that are further described in the following detailed descriptions. This summary is not intended to identify key features or essential features of the claimed subject matter, nor is it to be construed as limiting the scope of the claimed subject matter.

Disclosed herein is a mask for use in providing oxygen or nebulized medicaments. The mask includes a plurality of apertures configured to receive at least two straps in at least three different positions along a vertical length of the mask. The mask also includes a fitter configured for conforming to a bridge of a nose, at least one one-way valve, at least one exhalation port, and an inhalation port for receiving the oxygen or nebulized medicaments. In operation an oxygen delivery tube and reservoir bag are connected to the inhalation port.

According to one or more embodiments, the mask defines six apertures configured for receiving two straps.

According to one or more embodiments is a mask. The mask defines a plurality of apertures configured to receive at least two straps. The mask also includes a fitter, configured for conforming to a patient's nose. The mask also includes at least one one-way valve. The mask includes an inhalation port and defines at least one exhalation port. In operation, an oxygen delivery tube and a reservoir bag are connected to the inhalation port.

According to one or more embodiments, the mask defines four apertures configured for receiving two straps.

According to one or more embodiments the straps are made from elastic formed from rubber threads wrapped in a secondary fiber such as cotton, polyester, nylon, or a blend thereof.

According to one or more embodiments, the fitter is made of metal.

According to one or more embodiments, the mask includes at least two tabs attached to an outer circumference of the mask.

According to one or more embodiments, the mask includes wings fixedly attached to and extending outwardly from two sides of the mask, where the wings define apertures for receiving the at least two straps.

According to one or more embodiments, the apertures are substantially circular in shape.

According to one or more embodiments, the apertures are substantially rectangular in shape.

According to one or more embodiments, the at least two strap are made from a spandex-terylene material.

According to one or more embodiments, the mask defines eight apertures configured for receiving two straps.

According to one or more embodiments, the mask defines at least eight apertures configured for receiving two straps in multiple positions along the vertical length of the mask.

According to one or more embodiments, is a mask for use in providing oxygen or nebulized medicaments. The mask includes at least two straps and a plurality of apertures defined along two sides of the mask. The apertures are configured for receiving the at least two straps at more than two positions along a length of the mask. The mask includes at least one exhalation port and an inhalation port for receiving the oxygen or nebulized medicaments. In operation an oxygen delivery tube and reservoir bag are connected to the inhalation port.

According to one or more embodiments, the apertures defined by the mask are pairs of slots which run along the length of the two sides of the mask.

According to one or more embodiments, the apertures are substantially rectangular in shape.

According to one or more embodiments, the mask defines six apertures configured for receiving two straps.

According to one or more embodiments, the mask defines at least eight apertures configured for receiving two straps in multiple positions along the length of the mask.

According to one or more embodiments, the mask defines at least eight apertures, wherein the apertures are equidistantly spaced along the two sides of the mask and configured for receiving two straps in multiple positions along the length of the mask.

According to one or more embodiments, is a mask for use in providing oxygen or nebulized medicaments. The mask includes two wings. The wings extending outwardly, and substantially perpendicularly, from two sides of the mask. The mask also includes a plurality of apertures defined within the two wings. The apertures are configured for receiving at least two straps at more than two positions along a length of the mask. The mask includes at least one exhalation port, and an inhalation port for receiving the oxygen or nebulized medicaments. In operation an oxygen delivery tube and reservoir bag are connected to the inhalation port.

According to one or more embodiments, each wing defines at least eight apertures, equidistantly spaced within each wing and configured for receiving two straps in multiple positions along the length of the mask.

### BRIEF DESCRIPTION OF THE DRAWINGS

The foregoing, as well as the following Detailed Description of preferred embodiments, is better understood when read in conjunction with the appended drawings. For the purposes of illustration, there is shown in the drawings exemplary embodiments; however, the presently disclosed subject matter is not limited to the specific methods and instrumentalities disclosed.

The embodiments illustrated, described, and discussed herein are illustrative of the present invention. As these embodiments of the present invention are described with reference to illustrations, various modifications or adaptations of the methods and or specific structures described may become apparent to those skilled in the art. It will be appreciated that modifications and variations are covered by the above teachings and within the scope of the appended claims without departing from the spirit and intended scope thereof. All such modifications, adaptations, or variations that rely upon the teachings of the present invention, and through which these teachings have advanced the art, are considered to be within the spirit and scope of the present invention. Hence, these descriptions and drawings should not be considered in a limiting sense, as it is understood that the present invention is in no way limited to only the embodiments illustrated.

FIG. 1 is a front facing view of a mask, according to one or more embodiments of the presently disclosed subject matter.

FIG. 2 is a perspective view of the mask, according to one or more embodiments of the presently disclosed subject matter.

FIG. 3 is a side view of the mask, according to one or more embodiments of the presently disclosed subject matter.

FIG. 4 is a bottom facing view of the mask, according to one or more embodiments of the presently disclosed subject matter.

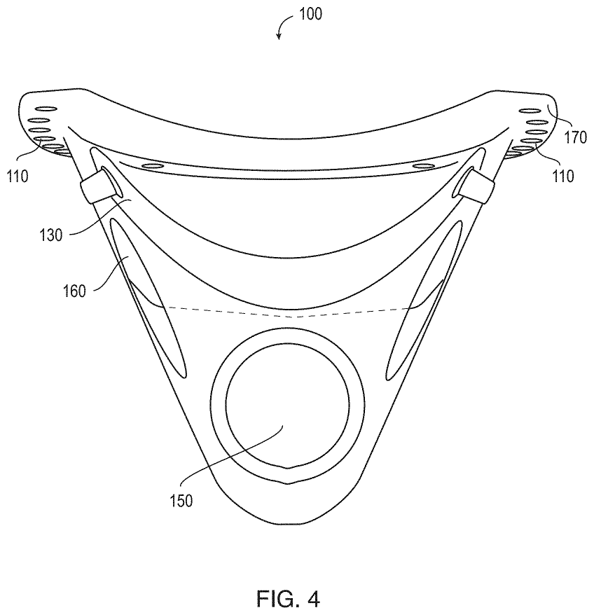

FIG. 5 is a side view of the mask incorporating two straps, according to one or more embodiments of the presently disclosed subject matter.

FIG. 6 is a perspective view of the mask incorporating two straps, according to one or more embodiments of the presently disclosed subject matter.

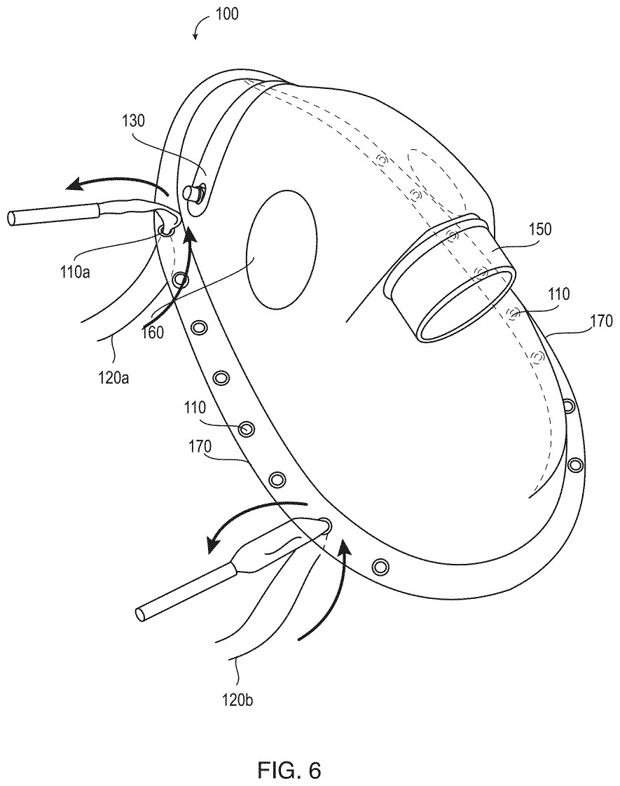

FIG. 7 is a perspective view of the mask incorporating two straps, according to one or more embodiments of the presently disclosed subject matter.

FIG. 8 is a front facing view of the mask, according to one or more embodiments of the presently disclosed subject matter.

FIG. 9 is a perspective view of the mask, according to one or more embodiments of the presently disclosed subject matter.

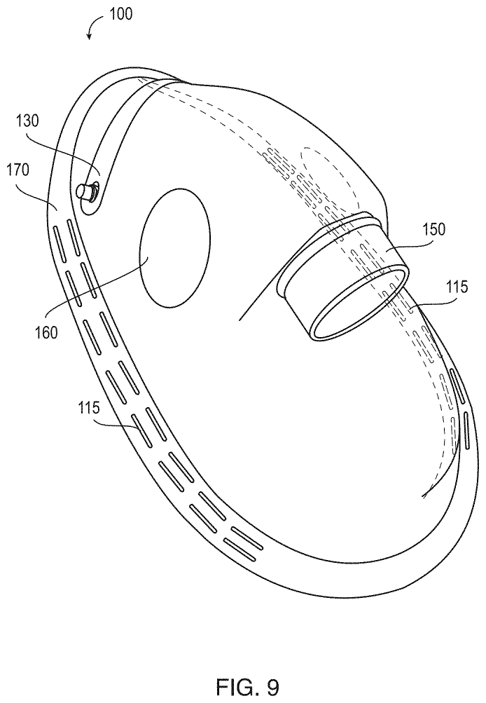

FIG. 10 is a side view of the mask, according to one or more embodiments of the presently disclosed subject matter.

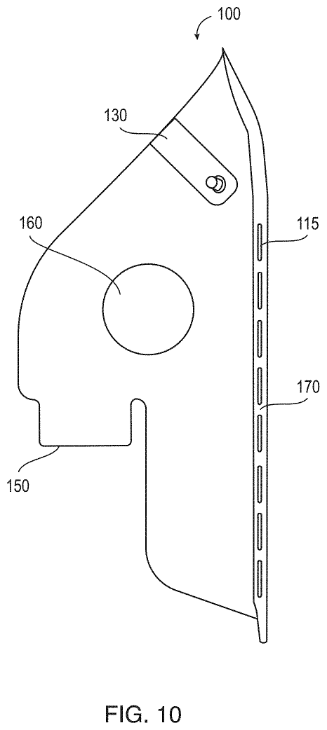

FIG. 11 is a bottom facing view of the mask, according to one or more embodiments of the presently disclosed subject matter.

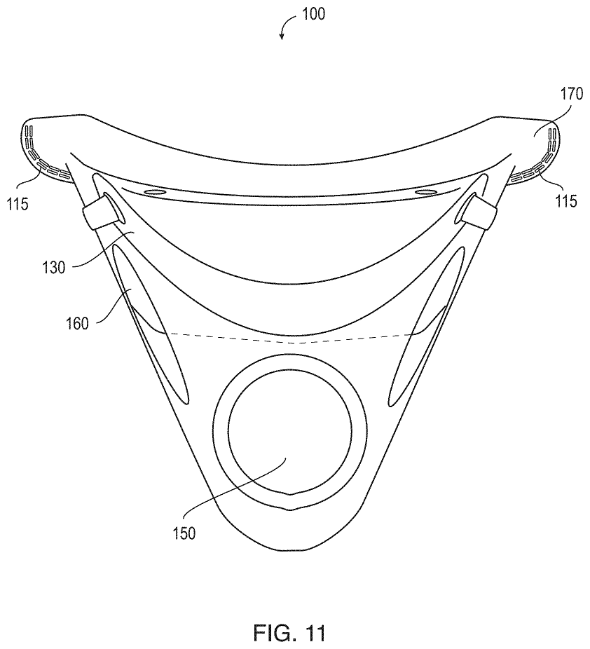

FIG. 12 is a side view of the mask incorporating two straps, according to one or more embodiments of the presently disclosed subject matter.

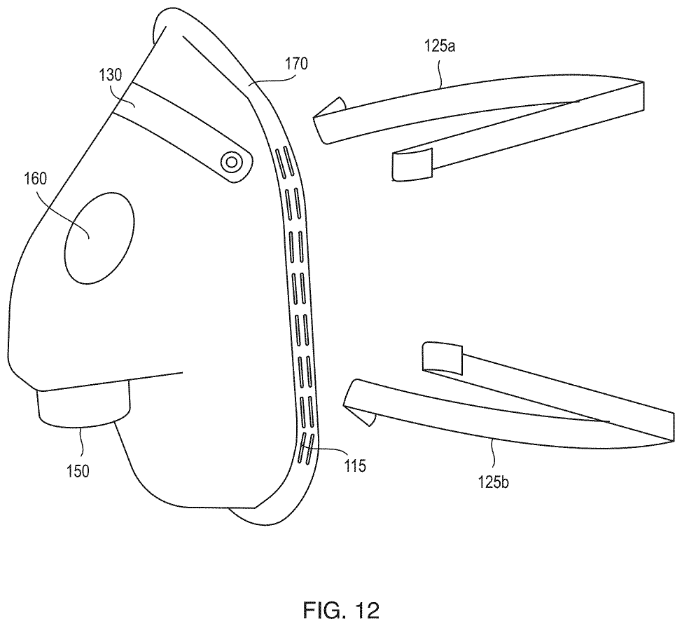

FIG. 13 is a perspective view of the mask incorporating two straps, according to one or more embodiments of the presently disclosed subject matter.

FIG. 14 is a perspective view of the mask incorporating two straps, according to one or more embodiments of the presently disclosed subject matter.

FIG. 15 is a front facing view of the mask incorporating one strap, according to one or more embodiments of the presently disclosed subject matter.

FIG. 16 is a front facing view of the mask incorporating one strap, according to one or more embodiments of the presently disclosed subject matter.

FIG. 17 is a front facing view of a mask with wings, according to one or more embodiments of the presently disclosed subject matter.

FIG. 18 is a front facing view of the mask with wings, according to one or more embodiments of the presently disclosed subject matter.

FIG. 19 is a perspective view of the mask in operation, according to one or more embodiments of the presently disclosed subject matter

### DETAILED DESCRIPTION

These descriptions are presented with sufficient details to provide an understanding of one or more particular embodiments of broader inventive subject matters. These descriptions expound upon and exemplify particular features of those particular embodiments without limiting the inventive subject matters to the explicitly described embodiments and features. Considerations in view of these descriptions will likely give rise to additional and similar embodiments and features without departing from the scope of the inventive subject matters. Although the term “step” may be expressly used or implied relating to features of processes or methods, no implication is made of any particular order or sequence among such expressed or implied steps unless an order or sequence is explicitly stated.

Any dimensions expressed or implied in the drawings and these descriptions are provided for exemplary purposes. Thus, not all embodiments within the scope of the drawings and these descriptions are made according to such exemplary dimensions. The drawings are not made necessarily to scale. Thus, not all embodiments within the scope of the drawings and these descriptions are made according to the apparent scale of the drawings with regard to relative dimensions in the drawings. However, for each drawing, at least one embodiment is made according to the apparent relative scale of the drawing.

Unless defined otherwise, all technical and scientific terms used herein have the same meaning as commonly understood to one of ordinary skill in the art to which the presently disclosed subject matter pertains. Although any methods, devices, and materials similar or equivalent to those described herein can be used in the practice or testing of the presently disclosed subject matter, representative methods, devices, and materials are now described.

Following long-standing patent law convention, the terms “a”, “an”, and “the” refer to “one or more” when used in the subject specification, including the claims. Thus, for example, reference to “a device” can include a plurality of such devices, and so forth.

Unless otherwise indicated, all numbers expressing quantities of components, conditions, and so forth used in the specification and claims are to be understood as being modified in all instances by the term “about”. Accordingly, unless indicated to the contrary, the numerical parameters set forth in the instant specification and attached claims are approximations that can vary depending upon the desired properties sought to be obtained by the presently disclosed subject matter.

As used herein, the term “about”, when referring to a value or to an amount of mass, weight, time, volume, concentration, and/or percentage can encompass variations of, in some embodiments +/−20%, in some embodiments +/−10%, in some embodiments +/−5%, in some embodiments +/−1%, in some embodiments +/−0.5%, and in some embodiments +/−0.1%, from the specified amount, as such variations are appropriate in the presently disclosed subject matter.

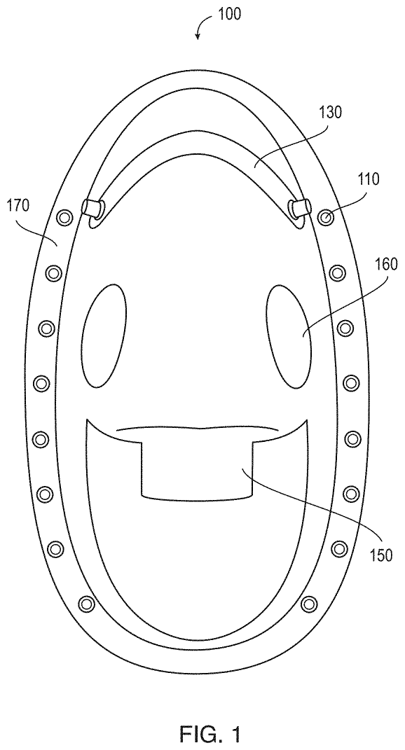

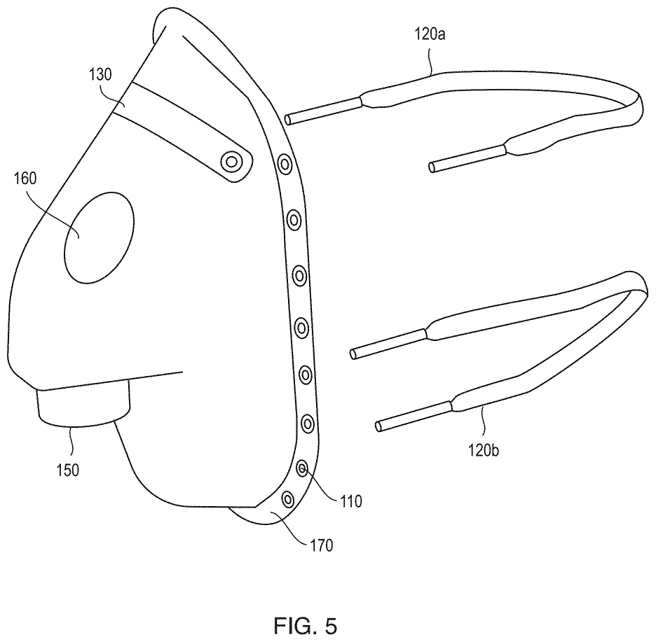

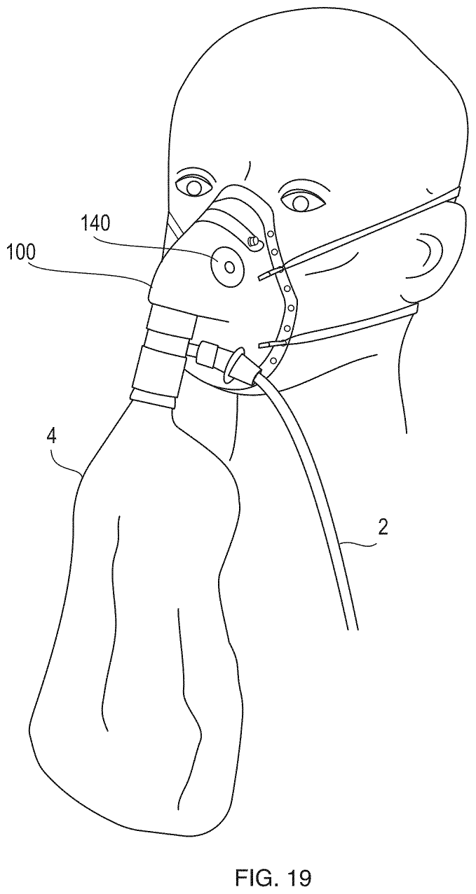

At least one embodiment of the presently disclosed subject matter is illustrated throughout the drawings and in particular reference to FIG. 1. Disclosed herein is a mask 100. The mask defines a plurality of apertures 110 configured to receive at least two straps 120 as shown in FIG. 5. The mask may also include a fitter 130, configured for conforming to a shape of a patient's nose. The mask also includes an inhalation port 150 and defines at least one exhalation port 160. In operation, an oxygen delivery tube 2 and a reservoir bag 4 are connected to the inhalation port 150 as seen in FIG. 19.

As shown in FIG. 19, the mask may also include at least one one-way valve 140. The one-way valve 140 may be used interchangeably with the exhalation port 160 of FIG. 1. If the one-way valve 140 is substituted for the exhalation port, it is configured such that air can only leave the mask (one-way out). A one-way valve, that is only one-way out, is a type of exhalation port. Additionally there may also be a one-way valve (not shown) inside of the inhalation port 150. The one-way valve inside the inhalation port is configured to only allow air into the mask (one-way in).

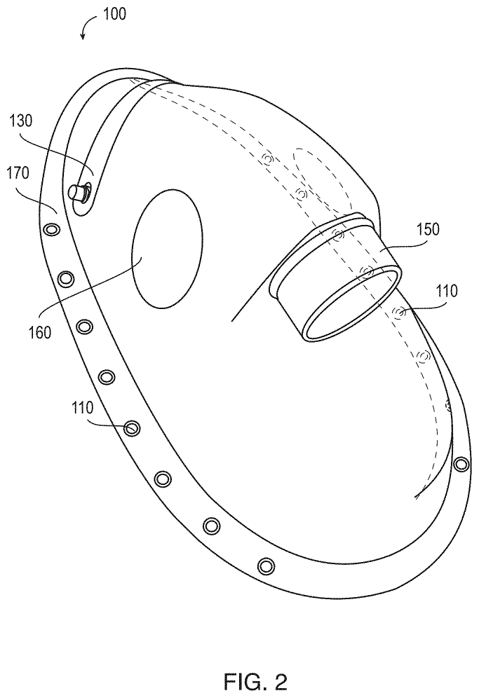

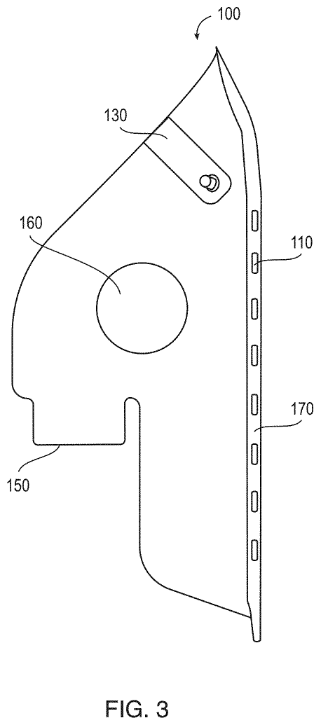

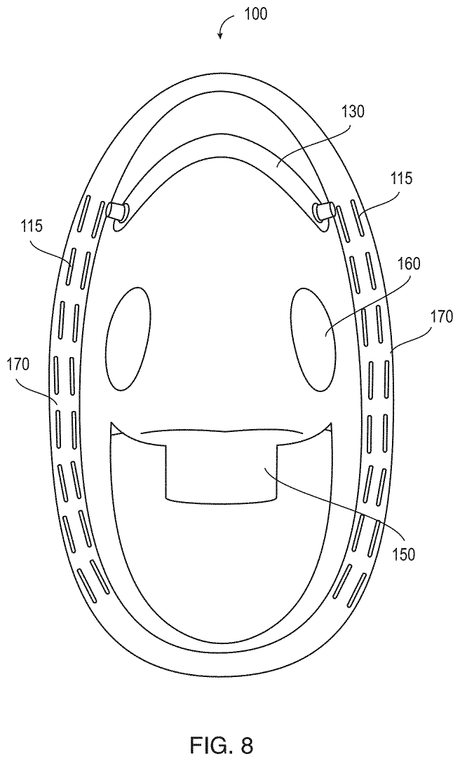

Turning to FIG. 2, the mask 100 defines a plurality of equidistantly spaced apertures 110 that run along each side 170 of the mask 100. The apertures 110 may be substantially circular in shape as illustrated in FIG. 2. Alternatively, the apertures 110 may be substantially rectangular as seen in FIG. 3. The apertures may also be pairs of slots 115, as illustrated, for example, in FIG. 8. The apertures do not have to be equidistantly spaced along each side, but equidistant spacing may provide the most range of options for strap placement.

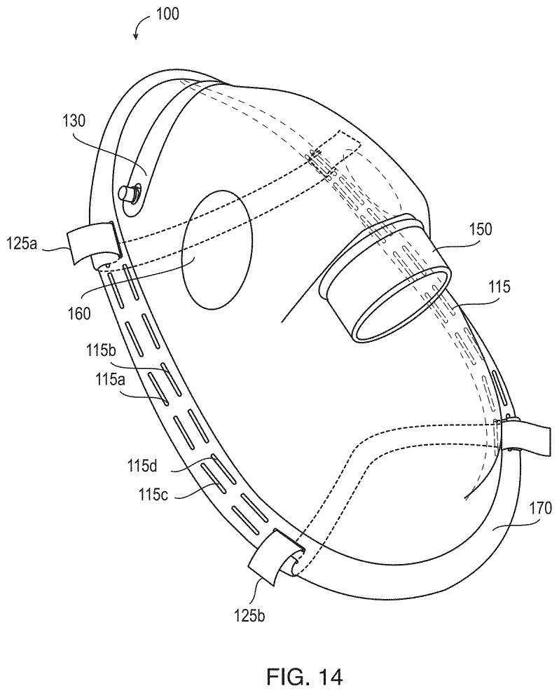

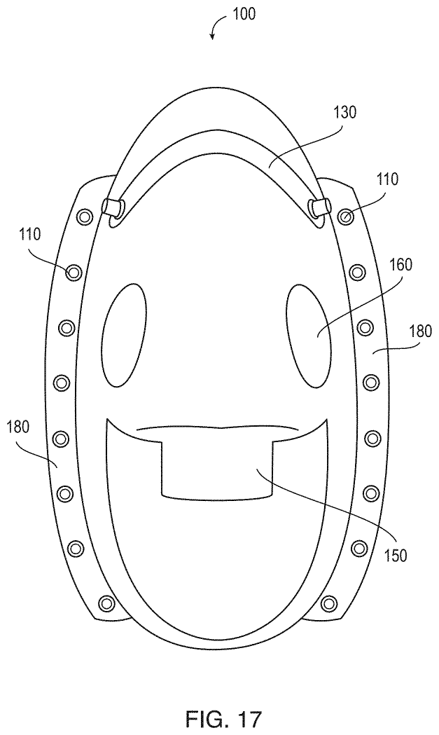

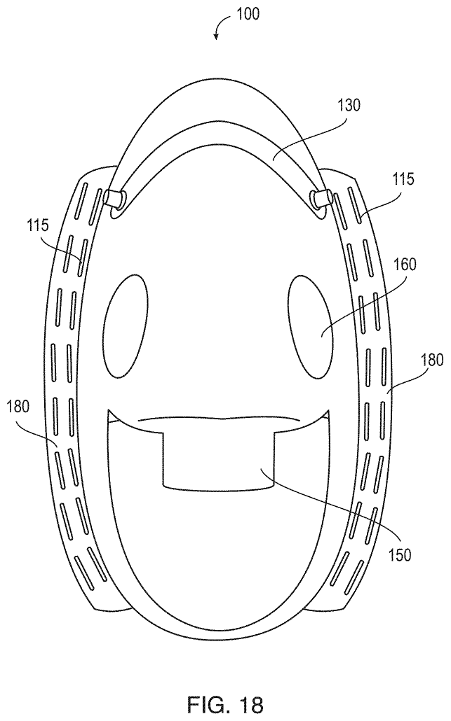

The slots 115 can be formed into the sides of the mask 170 as illustrated in FIG. 14. Alternatively, the slots 115 can be formed into attached tabs or wings 180, which extend outwardly from the edges of the mask as illustrated in FIG. 18. The use of wings 180 may add comfort by preventing abrasion of the strap ends with a patient's face. The use of wings 180 may also allow the mask 100 to sit more flush against the patient's face. Wings 180 may be used with circular apertures 110 as shown in FIG. 17 or with pairs of slots 115 as shown in FIG. 18. Manufacturing preferences may determine the placement of the slots 115.

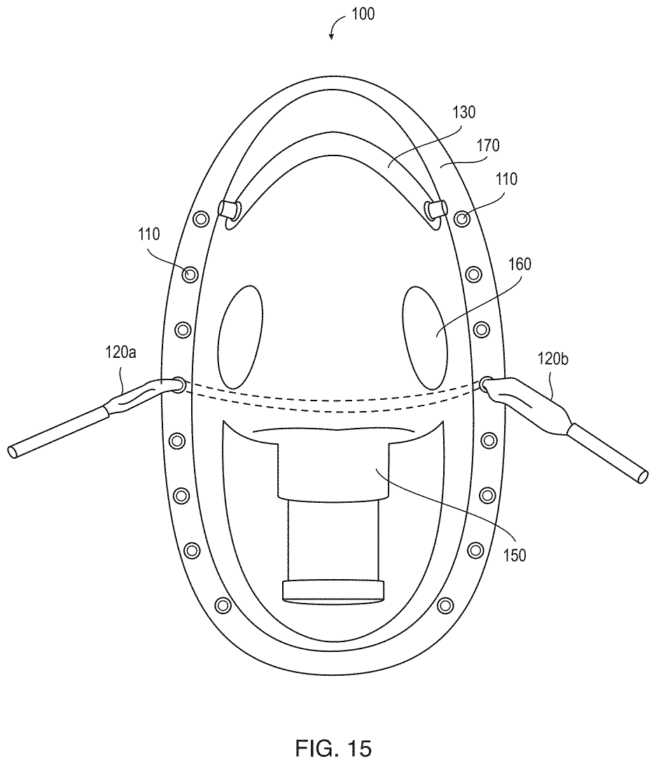

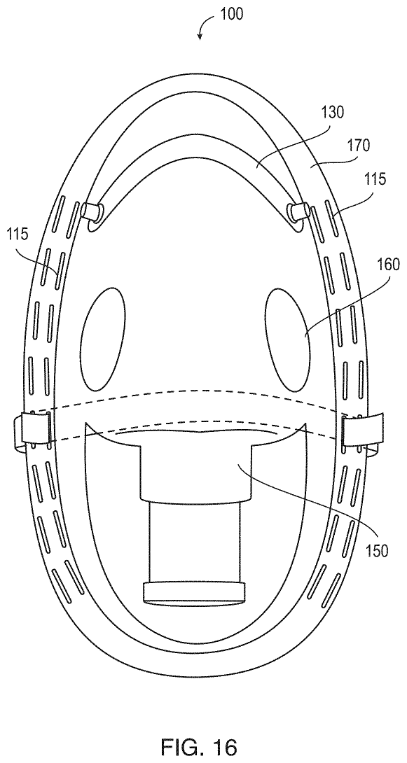

In some instances, circular apertures 110, as illustrated in FIG. 15, can be preferable to slots 115, as illustrated in FIG. 16, because circular apertures are more easily reinforced, and may be less prone to tearing. According to other embodiments, slots 115 are preferable to circular apertures 110 because slots can accommodate flat straps 125a and 125b as illustrated in FIG. 14. The flat straps 125 may be more comfortable and less prone to slipping on a patient's head.

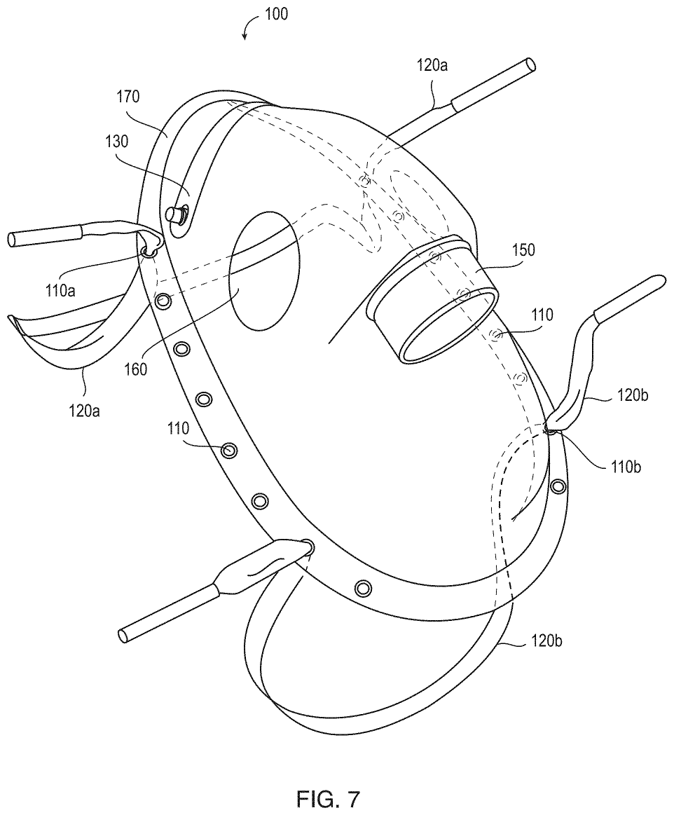

This design of mask 100 provides a more stable and comfortable means of supplying oxygen and/or aerosolized or nebulized medications than what is currently available. This is accomplished by providing a continuous row of apertures 110 along each side 170 of the mask 100, which provide multiple sites of adjustment as illustrated in FIG. 7. By using at least two elastic straps 120, at multiple sites of adjustment, as illustrated in FIG. 7, the mask 100 can accommodate varied facial contours and be positioned for the best fit. The elastic straps 120 can then be pulled to the optimal tension to secure the mask 100 to the individual's face and loosened as appropriate for comfort and fit.

There are many advantages to being able to adjust the positioning of straps 120 horizontally and vertically along each side. For example, in FIG. 7 shoestring style straps 120a and 120b can be independently tightened. Straps 120a and 120b can also be spaced closer together, or moved further apart depending on the size and shape of the patient's head. Many physical variations exist amongst patients, including different facial contours and sizes, differences in individual anatomy including ear size or shape, and the presence of wounds or injuries on the head of an individual where contact must be avoided. A better fitting mask with highly individualized adjustment features can help to prevent skin breakdown on the ears and nose that is observed with poorly fitting and sliding masks.

Individuals are likely to better tolerate the administration of oxygen and medications with a mask that fits securely and does not slide around or cut across their ears, and can be adjusted in multiple ways for optimal comfort and fit. Hospitals seeking masks that minimize hospital acquired wounds will view this design as an improvement over current designs. This is important, considering current regulations place the financial burden of treating hospital acquired wounds on the hospital. Another cost consideration is that the mask of the present disclosure will not be significantly more expensive to produce, and therefore, could be competitively priced whilst having additional beneficial features.

Patients, as well as individuals in a home setting, are much more likely to comply with treatment plans if their mask does not cause discomfort. There has been a long felt need in the industry for a mask that fits securely and minimizes wounding, while avoiding sensitive areas of the face and neck. The presently disclosed subject matter accomplishes these objectives.

The mask 100 can be constructed using various methods. Multiple variations exist to attach the straps along the sides of the mask. One variation uses apertures in the form of reinforced holes or circular apertures 110 along the outer side edges 170 of the mask 100 as illustrated in multiple figures including FIG. 7. These apertures 110 will extend vertically along each outer side edge 170 of the mask to allow for maximum adjustment points for placing straps 120. The straps 120 are threaded in, pulled through, and adjusted to fit depending on individual requirements.

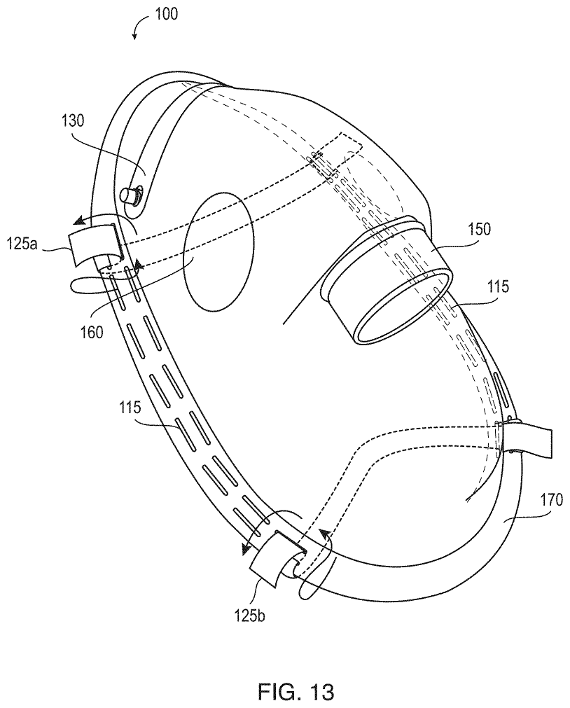

In another embodiment, the apertures are in the form of pairs of slots 115 as illustrated in FIG. 8. The slots 115 are configured in the same manner and in the same locations as the circular apertures and run along the length of two sides 170 of the mask 100. In the slot variation, as illustrated in FIGS. 13 and 14, the strap 125a or 125b is threaded into one slot 115c and out of its pair slot 115d for adjustment and to secure the strap as illustrated in FIG. 13. This allows for placement of the straps 125 in multiple positions along the vertical length of the mask 100.

Another embodiment uses pairs of slots 115, as previously described, except the slots are defined along each outer side edge on wings 180 as illustrated in FIG. 18. The wings 180 are made from the same material as the mask 100. This embodiment can allow for the wings 180 with slots 115 to be made separately, and fused to the mask 100 during production. The embodiment of FIG. 18 allows additional space for threading the straps 125 (not shown in FIG. 18).

According to one or more embodiments of the presently disclosed subject matter, the straps threaded through the apertures 110 of the mask 100 are shoestring elastic straps 120 as illustrated for example in FIG. 7. The tip of this type of strap 120 facilitates insertion through the apertures 110. The tip prevents fraying of the strap and allows for a thicker strap to be threaded through the smaller hole or slot and provides a method to secure the strap 120 to the mask. The elastic properties of this type of strap 120 are advantageous because of the ability to pull the strap 120 tighter or loosen it quickly depending on individual requirements.

The number of straps can be chosen depending on individual requirements. At least two straps may provide an optimal fit if threaded through the apertures at points which best fit the individual. One strap may be used as well, but is not the optimal option available with the mask of the present disclosure.

According to one or more embodiments, the mask 100 can be made from one or more of any substances known or used in the art, including but not limited to, soft, transparent PVC plastic and vinyl. The mask 100 may be made from a single piece of plastic. The mask can generally be made from a semi-flexible material, and because of the various strap positions the mask can accommodate, the mask 100 requires no other adjustable portions or components.

The features of the mask 100 described herein may be applied to any full or half face mask including adult or pediatric masks. A half mask is similar in function to a full mask, except it does not go down over the chin and mouth; it mostly covers the nose.

Particular embodiments and features have been described with reference to the drawings. It is to be understood that these descriptions are not limited to any single embodiment or any particular set of features, and that similar embodiments and features may arise or modifications and additions may be made without departing from the scope of these descriptions and the spirit of the appended claims.

These and other changes can be made to the disclosure in light of the above Detailed Description. While the above description describes certain embodiments of the disclosure, and describes the best mode contemplated, no matter how detailed the above appears in text, the teachings can be practiced in many ways. Details of the system may vary considerably in its implementation details, while still being encompassed by the subject matter disclosed herein. As noted above, particular terminology used when describing certain features or aspects of the disclosure should not be taken to imply that the terminology is being redefined herein to be restricted to any specific characteristics, features, or aspects of the disclosure with which that terminology is associated. In general, the terms used in the following claims should not be construed to limit the disclosure to the specific embodiments disclosed in the specification, unless the above Detailed Description section explicitly defines such terms. Accordingly, the actual scope of the disclosure encompasses not only the disclosed embodiments, but also all equivalent ways of practicing or implementing the disclosure under the claims.

What is claimed:

1. A mask for use in providing oxygen or nebulized medicaments, the mask comprising:
a plurality of apertures configured to receive at least two straps in at least three different positions along a vertical length of the mask;
a fitter configured for conforming to a bridge of a nose;
at least one one-way valve;
at least one exhalation port; and
an inhalation port for receiving the oxygen or nebulized medicaments,
wherein, in operation an oxygen delivery tube and reservoir bag are connected to the inhalation port.

2. The mask of claim 1, wherein the mask defines six apertures configured for receiving two straps.

3. The mask of claim 1, wherein the straps are made from elastic formed from rubber threads wrapped in a secondary fiber such as cotton, polyester, nylon, or a blend thereof.

4. The mask of claim 1, wherein the fitter is made of metal.

5. The mask of claim 1, further comprising at least two tabs attached to an outer circumference of the mask.

6. The mask of claim 1, further comprising wings fixedly attached to and extending outwardly from two sides of the mask, wherein the wings define apertures for receiving the at least two straps.

7. The mask of claim 1, wherein the apertures are substantially circular in shape.

8. The mask of claim 1, wherein the apertures are substantially rectangular in shape.

9. The mask of claim 1, wherein the at least two straps are made from a spandex-terylene material.

10. The mask of claim 1, wherein the mask defines at least eight apertures configured for receiving two straps in multiple positions along the vertical length of the mask.

11. A mask for use in providing oxygen or nebulized medicaments, the mask comprising:
at least two straps;
a plurality of apertures defined along two sides of the mask,
wherein the apertures are configured for receiving the at least two straps at more than two positions along a length of the mask;
at least one exhalation port; and
an inhalation port for receiving the oxygen or nebulized medicaments,
wherein, in operation an oxygen delivery tube and reservoir bag are connected to the inhalation port.

12. The mask of claim 11, wherein the apertures defined by the mask are pairs of slots which run along the length of the two sides of the mask.

13. The mask of claim 11, wherein the apertures are substantially rectangular in shape.

14. The mask of claim 11, wherein the mask defines six apertures configured for receiving two straps.

15. The mask of claim 11, wherein the mask defines at least eight apertures, wherein the apertures are equidistantly spaced along the two sides of the mask and configured for receiving two straps in multiple positions along the length of the mask.

16. A mask for use in providing oxygen or nebulized medicaments, the mask comprising:
two wings, the wings extending outwardly, and substantially perpendicularly, from two sides of the mask;
a plurality of apertures defined within the two wings,
wherein the apertures are configured for receiving at least two straps at more than two positions along a length of the mask;
at least one exhalation port; and
an inhalation port for receiving the oxygen or nebulized medicaments,
wherein, in operation an oxygen delivery tube and reservoir bag are connected to the inhalation port.

17. The mask of claim 16, wherein the apertures defined by the mask are pairs of slots which run along the length of the two sides of the mask.

18. The mask of claim 16, wherein the apertures are substantially rectangular in shape.

19. The mask of claim 16, wherein the mask defines six apertures configured for receiving two straps.

20. The mask of claim 16, wherein each wing defines at least eight apertures, equidistantly spaced within each wing and configured for receiving two straps in multiple positions along the length of the mask.
